SEQUENCE LISTING (1 sequence)

SEQ ID NO: 1 - DNA, 1415 bp, synthetic construct
  source 1..1415: mol_type = other DNA
catgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacccttgtggggggagccgtctaaggt

(19) United States
(12) Patent Application Publication
(10) Pub. No.: US 2026/0061011 A1
(43) Pub. Date: Mar. 5, 2026

(54) BIFIDOBACTERIUM ANIMALIS SUBSP. LACTIS FOR REDUCING ABETA42 DEPOSITION AND AN APPLICATION THEREOF

(71) Applicant: Tianjin InnoOrigin Biological Technology Co., Ltd., TIANJIN (CN)

(72) Inventors: Wu LIANG, TIANJIN (CN); Haikuan WANG, TIANJIN (CN); Xuemei HAN, TIANJIN (CN); Longgang JIA, TIANJIN (CN)

(21) Appl. No.: 19/063,329

(22) Filed: Feb. 26, 2025

(30) Foreign Application Priority Data
Sep. 4, 2024 (CN) 202411230717.5

(51) Int. Cl. A61K 35/745; A61K 9/19; A61P 25/28; C12N 1/20; C12R 1/01

(52) U.S. Cl. CPC A61K 35/745; A61K 9/19; A61P 25/28; C12N 1/205; C12R 2001/01

(57) ABSTRACT

A Bifidobacterium animalis subsp. lactis for reducing Abeta42 deposition and an application, a name of the Bifidobacterium animalis subsp. lactis is Bifidobacterium animalis subsp. lactis IOB-LO7, and classified as Bifidobacterium animalis subsp. lactis, the Bifidobacterium animalis subsp. lactis IOB-LO7 is preserved in the General Microbiology Center of the China General Microbiological Culture Collection Center (CGMCC) on Dec. 23, 2021, with the collection number of CGMCC No. 24185. By reducing the levels of Aβ42 in the cerebral cortex and hippocampus, clearing Aβ amyloid plaques, improving communication between neurons, reducing brain neuroinflammation, and protecting nerve cells, it helps to restore cognitive function and improve Alzheimer's disease. Additionally, it can also improve gut microbiota imbalance caused by Alzheimer's disease.

### CROSS REFERENCE TO RELATED APPLICATIONS

This application claims foreign priority of Chinese Patent Application No. 202411230717.5, filed on Sep. 4, 2024 in the China National Intellectual Property Administration, the disclosures of all of which are hereby incorporated by reference.

### TECHNICAL FIELD

The present disclosure relates to the microbiology technical field, in particular to a Bifidobacterium animalis subsp. lactis for reducing Abeta42 deposition and an application thereof.

### SEQUENCE LISTING

The present application contains a sequence listing which was filed electrically in XML format and is hereby incorporated by reference in its entirety. The XML-format sequence listing file was created on Apr. 16, 2025, named as “SEQUENCE LISTING”, and is 13.9 kb in size.

### BACKGROUND

Alzheimer's disease (AD) is a common chronic degenerative disease of central nervous system, accounting for 60%-70% of all dementia cases. AD patients typically exhibit symptoms such as cognitive impairment, behavioral disturbances, memory loss, language difficulties, and personality changes. In China, the number of dementia patients is extremely high, with approximately 10-11 million patients aged 60 and above, and 60%-80% of them suffering from Alzheimer's disease.

The onset of Alzheimer's disease is caused by various types of neuronal damage, including the aggregation of amyloid beta (Aβ or Abeta), hyperphosphorylation of tau proteins, oxidative stress, and inflammatory responses. The neuropathological features of Alzheimer's disease primarily include extracellular amyloid plaques (SP), intracellular neurofibrillary tangles (NFTs), loss of synapses, and exacerbated neuroinflammatory responses mediated by microglia and astrocytes.

Amyloid protein deposition is the core pathological feature of Alzheimer's disease. Its accumulation in the brain forms plaques, which trigger a series of neuroinflammatory responses. Additionally, through the bidirectional interaction between the brain and the gut, amyloid deposition can alter the structure of the gut microbiota. In recent years, a hypothesis has emerged suggesting that the onset of Alzheimer's disease may be linked to dysbiosis of the gut microbiota. Disruption of the gut microbiome can lead to an imbalance in microbial metabolites, such as lipopolysaccharides (LPS) and bacterial amyloids. These microbial products can damage the intestinal barrier, increasing intestinal permeability. Bacterial-derived LPS and amyloids can cross the compromised intestinal mucosa, enter the bloodstream, and reach the central nervous system, promoting the nucleation of Aβ aggregates and triggering inflammatory responses. This process may damage the blood-brain barrier, leading to neuroinflammation and neuronal damage, thereby causing neurodegeneration and contributing to the development of Alzheimer's disease.

Probiotics have been shown to restore the homeostasis of the gut microbiota and slow the progression of Alzheimer's disease, particularly by reducing inflammation and improving cognitive decline. Inactivated probiotics can influence the host's intestinal microbiota by stimulating the mucosal immune response. Therefore, intervention through probiotics or postbiotics is considered one of the best measures to prevent cognitive decline in Alzheimer's disease.

### SUMMARY

In view of the technical problems existing in the existing technology, the present disclosure aims to provide a Bifidobacterium animalis subsp. lactis for reducing Abeta42 deposition and an application thereof. Specifically, provides a Bifidobacterium animalis subsp. lactis IOB-LO7, classified as Bifidobacterium animalis subsp. lactis, Bifidobacterium animalis subsp. lactis IOB-LO7 bacterial powders, Bifidobacterium animalis subsp. lactis IOB-LO7 postbiotics, or pharmaceutical for protecting nerve cells. The present disclosure also provide an application of a Bifidobacterium animalis subsp. lactis IOB-LO7 for reducing Abeta42 deposition.

The present disclosure provides a Bifidobacterium animalis subsp. lactis for reducing Abeta42 deposition, wherein, a name of the Bifidobacterium animalis subsp. lactis is Bifidobacterium animalis subsp. lactis IOB-LO7, and classified as Bifidobacterium animalis subsp. lactis, the Bifidobacterium animalis subsp. lactis IOB-LO7 is preserved in the General Microbiology Center of the China General Microbiological Culture Collection Center (CGMCC) on Dec. 23, 2021, with the collection number of CGMCC No. 24185.

Specifically, a 16srDNA sequence of the Bifidobacterium animalis subsp. lactis for reducing Abeta42 deposition is:

(SEQ ID NO. 1)
CATGCAAGTCGAACGGGATCCCTGGCAGCTTGCTGTCGGGGTGAGAGTG
GCGAACGGGTGAGTAATGCGTGACCAACCTGCCCTGTGCACCGGAATAG
CTCCTGGAAACGGGTGGTAATACCGGATGCTCCGCTCCATCGCATGGTG
GGGTGGGAAATGCTTTTGCGGCATGGGATGGGGTCGCGTCCTATCAGCT
TGTTGGCGGGGTGATGGCCCACCAAGGCGTTGACGGGTAGCCGGCCTGA
GAGGGTGACCGGCCACATTGGGACTGAGATACGGCCCAGACTCCTACGG
GAGGCAGCAGTGGGGAATATTGCACAATGGGCGCAAGCCTGATGCAGCG
ACGCCGCGTGCGGGATGGAGGCCTTCGGGTTGTAAACCGCTTTTGTTCA
AGGGCAAGGCACGGTTTCGGCCGTGTTGAGTGGATTGTTCGAATAAGCA
CCGGCTAACTACGTGCCAGCAGCCGCGGTAATACGTAGGGTGCGAGCGT
TATCCGGATTTATTGGGCGTAAAGGGCTCGTAGGCGGTTCGTCGCGTCC
GGTGTGAAAGTCCATCGCCTAACGGTGGATCTGCGCCGGGTACGGGCGG
GCTGGAGTGCGGTAGGGGAGACTGGAATTCCCGGTGTAACGGTGGAATG
TGTAGATATCGGGAAGAACACCAATGGCGAAGGCAGGTCTCTGGGCCGT
CACTGACGCTGAGGAGCGAAAGCGTGGGGAGCGAACAGGATTAGATACC
CTGGTAGTCCACGCCGTAAACGGTGGATGCTGGATGTGGGGCCCTTTCC
ACGGGTCCCGTGTCGGAGCCAACGCGTTAAGCATCCCGCCTGGGGAGTA
CGGCCGCAAGGCTAAAACTCAAAGAAATTGACGGGGGCCCGCACAAGCG
GCGGAGCATGCGGATTAATTCGATGCAACGCGAAGAACCTTACCTGGGC
TTGACATGTGCCGGATCGCCGTGGAGACACGGTTTCCCTTCGGGGCCGG
TTCACAGGTGGTGCATGGTCGTCGTCAGCTCGTGTCGTGAGATGTTGGG
TTAAGTCCCGCAACGAGCGCAACCCTCGCCGCATGTTGCCAGCGGGTGA
TGCCGGGAACTCATGTGGGACCGCCGGGGTCAACTCGGAGGAAGGTGGG
GATGACGTCAGATCATCATGCCCCTTACGTCCAGGGCTTCACGCATGCT
ACAATGGCCGGTACAACGCGGTGCGACACGGTGACGTGGGGCGGATCGC
TGAAAACCGGTCTCAGTTCGGATCGCAGTCTGCAACTCGACTGCGTGAA
GGCGGAGTCGCTAGTAATCGCGGATCAGCAACGCCGCGGTGAATGCGTT
CCCGGGCCTTGTACACACCGCCCGTCAAGTCATGAAAGTGGGTAGCACC
CGAAGCCGGTGGCCCGACCCTTGTGGGGGGAGCCGTCTAAGGT.

The present disclosure further provides an application of a Bifidobacterium animalis subsp. lactis IOB-LO7 for reducing Abeta42 deposition, the Bifidobacterium animalis subsp. lactis is applied for preparing Bifidobacterium animalis subsp. lactis IOB-LO7 bacterial powders, Bifidobacterium animalis subsp. lactis IOB-LO7 postbiotics, or pharmaceutical for protecting nerve cells.

Furthermore, the Bifidobacterium animalis subsp. lactis bacterial powders are prepared by the following steps: preparing fermentation broth; and centrifuging the fermentation broth at a speed of 4000 to 6000 rpm for 20 to 30 minutes, wherein a temperature of the centrifuge is reduced to 4° C. to 6° C. in advance; and collecting sediments and pre-freezing the sediments at −82° C. to −80° C. for 2 to 3 hours; and performing a freeze-drying at a temperature of −80° C. to −60° C. for 24 to 48 hours to obtain the Bifidobacterium animalis subsp. lactis IOB-LO7 postbiotics powders.

Furthermore, the Bifidobacterium animalis subsp. lactis IOB-LO7 postbiotics are prepared by the following steps: preparing the fermentation broth; and centrifuging the fermentation broth at a speed of 4000 to 6000 rpm for 20 to 30 minutes, wherein a temperature of the centrifuge is reduced to 4° C. to 6° C. in advance; and collecting sediments and sterilizing the sediments at 80° C. to 95° C. for 20 to 25 minutes; pre-freezing the sediments at −82° C. to −80° C. for 2 to 3 hours, and freeze-drying the sediments to obtain powers of the Bifidobacterium animalis subsp. lactis IOB-LO7 postbiotics, wherein a drying temperature is set to −80° C. to −60° C., and a drying time is 24 to 48 hours.

Furthermore, the fermentation broth is prepared by the following steps: inoculating the Bifidobacterium animalis subsp. lactis IOB-LO7, stored in cryovials, into 15 to 20 mL of TPY fluid medium; and culturing for 18 to 24 hours in a strictly anaerobic environment at a culturing temperature of 35° C. to 38° C., to obtain first generation activated bacterial solution; and inoculating the first generation activated bacterial solution into 50 to 60 mL of the TPY fluid medium at an inoculation rate of 3% to 5%; and culturing for 18 to 24 hours in the strictly anaerobic environment at the culturing temperature of 35° C. to 38° C., to obtain second generation activated bacterial solution; and inoculating the second generation activated bacterial solution into 90 to 100 mL of the TPY fluid medium at an inoculation rate of 1% to 3%; and culturing for 18 to 20 hours in the strictly anaerobic environment at the culturing temperature of 35° C. to 38° C., to obtain fermented seed liquid; inoculating the fermented seed liquid into 800 to 1000 mL of the TPY fluid medium at the inoculation rate of 3% to 5%; and culturing for 18 to 20 hours in the strictly anaerobic environment at the culturing temperature of 35° C. to 38° C., to obtain the fermentation broth.

Furthermore, the pharmaceutical for protecting nerve cells includes the Bifidobacterium animalis subsp. lactis IOB-LO7, the Bifidobacterium animalis subsp. lactis IOB-LO7 powders, or the Bifidobacterium animalis subsp. lactis IOB-LO7 postbiotics.

Furthermore, a viable bacterial count of the Bifidobacterium animalis subsp. lactis IOB-LO7 powders is 1×10⁹ to 8×10¹¹ cfu/g, and a viable bacterial count of the pharmaceutical for protecting nerve cells is 1×10⁹ to 8×10¹¹ cfu/g.

The present invention provides a Bifidobacterium animalis subsp. lactis IOB-LO7, classified as Bifidobacterium animalis subsp. lactis. This strain reduces the levels of Aβ 42 by 42.1% to 64.9% in the cerebral cortex and hippocampus, significantly decreasing the accumulation of Aβ42 in brain tissue. On one hand, it clears Aβ amyloid plaques, greatly improving communication between neurons and helping to restore cognitive function. On the other hand, it reduces the brain inflammation caused by Aβ42 accumulation in brain tissue, thereby minimizing damage to nerve cells. Additionally, reducing Aβ42 concentration greatly improves synaptic transmission and accelerates the recovery of normal neural network function, effectively improving Alzheimer's disease. This strain also regulates the imbalance of gut microbiota metabolites, alleviates intestinal mucosal barrier damage in rats, enhances intestinal barrier permeability, prevents the release and spread of harmful metabolites such as LPS, reduces the nucleation of Aβ aggregates, thereby protecting the nervous system and reducing brain neuroinflammation.

### BRIEF DESCRIPTION OF THE DRAWINGS

FIG. 1 is a bar chart of a number of times of rats crossed a platform in different experimental groups according to an embodiment of the present disclosure.

FIG. 2 is a bar chart of an average speed of rats in an opening field test of different groups.

FIG. 3 is a bar chart of a total distance traveled by rats in the opening field test of different groups.

FIG. 4 is a bar chart of a total activity counts of rats in the opening field test of different experimental groups.

FIG. 5 is a bar chart of Aβ42 levels in hippocampus of rats in different groups.

FIG. 6 is a bar chart of Aβ42 levels in cerebral cortices of rats in different groups.

FIG. 7 is a bar chart of the relative area of Aβ42 plaque in the hippocampus of rats in different group.

FIG. 8 is a bar chart of relative transcription levels of TLR4 in different groups.

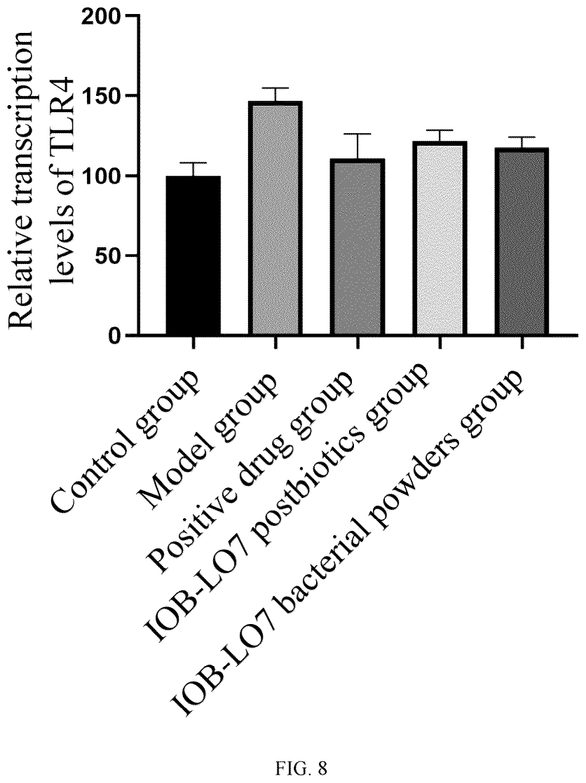

FIG. 9 is a bar chart of relative transcription levels of MyD88 in different groups.

FIG. 10 is a bar chart of relative transcription levels of NLRP3 in different groups.

FIG. 11 is a bar chart of relative transcription levels of IL-1β in different groups.

FIG. 12 is a bar chart of relative transcription levels of IL-18 in different groups.

FIG. 13 is a bar chart of LPS levels in rat feces in different groups.

FIG. 14 is a bar chart of LPS levels in rat serum in different groups.

FIG. 15 is a bar chart of LPS levels in the hippocampus of rats in different groups.

FIG. 16 is fluorescence imaging of pathological tissue sections of rat small intestine in different groups.

### DETAILED DESCRIPTION OF THE EMBODIMENTS

The technical solutions in the embodiments of the present disclosure will be described clearly and completely with reference to the accompanying drawings in the embodiments of the present disclosure. Obviously, the described embodiments are only a part of the embodiments of the present disclosure rather than all of them. Based on the embodiments in the present disclosure, all other embodiments obtained by those skilled in the art without creative work shall fall within the scope of protection of the present disclosure.

The present embodiment provides a Bifidobacterium animalis subsp. lactis for reducing Abeta42 deposition, a name of the Bifidobacterium animalis subsp. lactis is Bifidobacterium animalis subsp. lactis IOB-LO7, and classified as Bifidobacterium animalis subsp. lactis, the Bifidobacterium animalis subsp. lactis IOB-LO7 is preserved in the General Microbiology Center of the China General Microbiological Culture Collection Center (CGMCC) on Dec. 23, 2021, with the collection number of CGMCC No. 24185.

Specifically, a 16srDNA sequence of the Bifidobacterium animalis subsp. lactis for reducing Abeta42 deposition is:

(SEQ ID NO. 1)
CATGCAAGTCGAACGGGATCCCTGGCAGCTTGCTGTCGGGGTGAGAGTG
GCGAACGGGTGAGTAATGCGTGACCAACCTGCCCTGTGCACCGGAATAG
CTCCTGGAAACGGGTGGTAATACCGGATGCTCCGCTCCATCGCATGGTG
GGGTGGGAAATGCTTTTGCGGCATGGGATGGGGTCGCGTCCTATCAGCT
TGTTGGCGGGGTGATGGCCCACCAAGGCGTTGACGGGTAGCCGGCCTGA
GAGGGTGACCGGCCACATTGGGACTGAGATACGGCCCAGACTCCTACGG
GAGGCAGCAGTGGGGAATATTGCACAATGGGCGCAAGCCTGATGCAGCG
ACGCCGCGTGCGGGATGGAGGCCTTCGGGTTGTAAACCGCTTTTGTTCA
AGGGCAAGGCACGGTTTCGGCCGTGTTGAGTGGATTGTTCGAATAAGCA
CCGGCTAACTACGTGCCAGCAGCCGCGGTAATACGTAGGGTGCGAGCGT
TATCCGGATTTATTGGGCGTAAAGGGCTCGTAGGCGGTTCGTCGCGTCC
GGTGTGAAAGTCCATCGCCTAACGGTGGATCTGCGCCGGGTACGGGCGG
GCTGGAGTGCGGTAGGGGAGACTGGAATTCCCGGTGTAACGGTGGAATG
TGTAGATATCGGGAAGAACACCAATGGCGAAGGCAGGTCTCTGGGCCGT
CACTGACGCTGAGGAGCGAAAGCGTGGGGAGCGAACAGGATTAGATACC
CTGGTAGTCCACGCCGTAAACGGTGGATGCTGGATGTGGGGCCCTTTCC
ACGGGTCCCGTGTCGGAGCCAACGCGTTAAGCATCCCGCCTGGGGAGTA
CGGCCGCAAGGCTAAAACTCAAAGAAATTGACGGGGGCCCGCACAAGCG
GCGGAGCATGCGGATTAATTCGATGCAACGCGAAGAACCTTACCTGGGC
TTGACATGTGCCGGATCGCCGTGGAGACACGGTTTCCCTTCGGGGCCGG
TTCACAGGTGGTGCATGGTCGTCGTCAGCTCGTGTCGTGAGATGTTGGG
TTAAGTCCCGCAACGAGCGCAACCCTCGCCGCATGTTGCCAGCGGGTGA
TGCCGGGAACTCATGTGGGACCGCCGGGGTCAACTCGGAGGAAGGTGGG
GATGACGTCAGATCATCATGCCCCTTACGTCCAGGGCTTCACGCATGCT
ACAATGGCCGGTACAACGCGGTGCGACACGGTGACGTGGGGCGGATCGC
TGAAAACCGGTCTCAGTTCGGATCGCAGTCTGCAACTCGACTGCGTGAA
GGCGGAGTCGCTAGTAATCGCGGATCAGCAACGCCGCGGTGAATGCGTT
CCCGGGCCTTGTACACACCGCCCGTCAAGTCATGAAAGTGGGTAGCACC
CGAAGCCGGTGGCCCGACCCTTGTGGGGGGAGCCGTCTAAGGT.

A preferred embodiment of the present disclosure, the Bifidobacterium animalis subsp. lactis IOB-LO7 reduces the levels of Aβ42 in the cerebral cortex and hippocampus, clears Aβ amyloid plaques, improves communication between neurons, reduces brain neuroinflammation, protects nerve cells, helps restore cognitive function, and improves Alzheimer's disease.

The Bifidobacterium animalis subsp. lactis IOB-LO7 significantly reduces the accumulation of Aβ42 in brain tissue by lowering Aβ42 levels in the cerebral cortex and hippocampus. On one hand, it clears Aβ amyloid plaques, reduces direct neuronal damage, decreases oxidative stress and cell apoptosis, thereby improving the survival environment of neurons. This greatly enhances communication between neurons, helping to restore cognitive function. On the other hand, the Bifidobacterium animalis subsp. lactis IOB-LO7 reduces the activation of microglia, lowers the brain inflammation triggered by Aβ42 accumulation, inhibits inflammatory factors in the TLR4/MyD88/NLRP3 signaling pathway, and reduces damage to nerve cells. Additionally, reducing Aβ42 concentration significantly improves synaptic transmission and accelerates the recovery of normal neural network function, effectively improving Alzheimer's disease. The strain also regulates the imbalance of gut microbiota metabolites, alleviates intestinal mucosal barrier damage in rats, increases intestinal barrier permeability, prevents the release and spread of harmful metabolites such as LPS, reduces the nucleation of Aβ aggregates, and subsequently clears Aβ amyloid plaques, thereby protecting the nervous system and reducing brain neuroinflammation.

Another embodiment of the present disclosure provides an application of a Bifidobacterium animalis subsp. lactis IOB-LO7 for reducing Abeta42 deposition, the Bifidobacterium animalis subsp. lactis is applied for preparing Bifidobacterium animalis subsp. lactis IOB-LO7 bacterial powders, Bifidobacterium animalis subsp. lactis IOB-LO7 postbiotics, or pharmaceutical for protecting nerve cells.

In one embodiment, the Bifidobacterium animalis subsp. lactis bacterial powders are prepared by the following steps: preparing fermentation broth; and centrifuging the fermentation broth at a speed of 4000 to 6000 rpm for 20 to 30 minutes, wherein a temperature of the centrifuge is reduced to 4° C. to 6° C. in advance; and collecting sediments and pre-freezing the sediments at −82° C. to −80° C. for 2 to 3 hours; and performing a freeze-drying at a temperature of −80° C. to −60° C. for 24 to 48 hours to obtain the Bifidobacterium animalis subsp. lactis IOB-LO7 postbiotics powders.

In one embodiment, the Bifidobacterium animalis subsp. lactis IOB-LO7 postbiotics are prepared by the following steps: preparing the fermentation broth; and centrifuging the fermentation broth at a speed of 4000 to 6000 rpm for 20 to 30 minutes, wherein a temperature of the centrifuge is reduced to 4° C. to 6° C. in advance; and collecting sediments and sterilizing the sediments at 80° C. to 95° C. for 20 to 25 minutes; pre-freezing the sediments at −82° C. to −80° C. for 2 to 3 hours, and freeze-drying the sediments to obtain powers of the Bifidobacterium animalis subsp. lactis IOB-LO7 postbiotics, wherein a drying temperature is set to −80° C. to −60° C., and a drying time is 24 to 48 hours.

In one embodiment, the fermentation broth is prepared by the following steps: inoculating the Bifidobacterium animalis subsp. lactis IOB-LO7, stored in cryovials, into 15 to 20 mL of TPY fluid medium; and culturing for 18 to 24 hours in a strictly anaerobic environment at a culturing temperature of 35° C. to 38° C., to obtain first generation activated bacterial solution; and inoculating the first generation activated bacterial solution into 50 to 60 mL of the TPY fluid medium at an inoculation rate of 3% to 5%; and culturing for 18 to 24 hours in the strictly anaerobic environment at the culturing temperature of 35° C. to 38° C., to obtain second generation activated bacterial solution; and inoculating the second generation activated bacterial solution into 90 to 100 mL of the TPY fluid medium at an inoculation rate of 1% to 3%; and culturing for 18 to 20 hours in the strictly anaerobic environment at the culturing temperature of 35° C. to 38° C., to obtain fermented seed liquid; inoculating the fermented seed liquid into 800 to 1000 mL of the TPY fluid medium at the inoculation rate of 3% to 5%; and culturing for 18 to 20 hours in the strictly anaerobic environment at the culturing temperature of 35° C. to 38° C., to obtain the fermentation broth

In one embodiment, the TPY fluid medium was purchased from Haibo Biotechnology Co., Ltd., Qingdao High-tech Industrial Park, with the product number HBB570.

Specifically, the Bifidobacterium animalis subsp. lactis bacterial powders are prepared by the following steps: inoculating the Bifidobacterium animalis subsp. lactis IOB-LO7, stored in cryovials, into 15 to 20 mL of TPY fluid medium; and culturing for 18 to 24 hours in a strictly anaerobic environment at a culturing temperature of 35° C. to 38° C., to obtain first generation activated bacterial solution; and inoculating the first generation activated bacterial solution into 50 to 60 mL of the TPY fluid medium at an inoculation rate of 3% to 5%; and culturing for 18 to 24 hours in the strictly anaerobic environment at the culturing temperature of 35° C. to 38° C., to obtain second generation activated bacterial solution; and inoculating the second generation activated bacterial solution into 90 to 100 mL of the TPY fluid medium at an inoculation rate of 1% to 3%; and culturing for 18 to 20 hours in the strictly anaerobic environment at the culturing temperature of 35° C. to 38° C., to obtain fermented seed liquid; inoculating the fermented seed liquid into 800 to 1000 mL of the TPY fluid medium at the inoculation rate of 3% to 5%; and culturing for 18 to 20 hours in the strictly anaerobic environment at the culturing temperature of 35° C. to 38° C., to obtain the fermentation broth; and centrifuging the fermentation broth at a speed of 4000 to 6000 rpm for 20 to 30 minutes, wherein a temperature of the centrifuge is reduced to 4° C. to 6° C. in advance; and collecting sediments and pre-freezing the sediments at −82° C. to −80° C. for 2 to 3 hours; and performing a freeze-drying at a temperature of −80° C. to −60° C. for 24 to 48 hours to obtain the Bifidobacterium animalis subsp. lactis IOB-LO7 postbiotics powders.

The Bifidobacterium animalis subsp. lactis IOB-LO7 postbiotics are prepared by the following steps: inoculating the Bifidobacterium animalis subsp. lactis IOB-LO7, stored in cryovials, into 15 to 20 mL of TPY fluid medium; and culturing for 18 to 24 hours in a strictly anaerobic environment at a culturing temperature of 35° C. to 38° C., to obtain first generation activated bacterial solution; and inoculating the first generation activated bacterial solution into 50 to 60 mL of the TPY fluid medium at an inoculation rate of 3% to 5%; and culturing for 18 to 24 hours in the strictly anaerobic environment at the culturing temperature of 35° C. to 38° C., to obtain second generation activated bacterial solution; and inoculating the second generation activated bacterial solution into 90 to 100 mL of the TPY fluid medium at an inoculation rate of 1% to 3%; and culturing for 18 to 20 hours in the strictly anaerobic environment at the culturing temperature of 35° C. to 38° C., to obtain fermented seed liquid; inoculating the fermented seed liquid into 800 to 1000 mL of the TPY fluid medium at the inoculation rate of 3% to 5%; and culturing for 18 to 20 hours in the strictly anaerobic environment at the culturing temperature of 35° C. to 38° C., to obtain the fermentation broth; and centrifuging the fermentation broth at a speed of 4000 to 6000 rpm for 20 to 30 minutes, wherein a temperature of the centrifuge is reduced to 4° C. to 6° C. in advance; and collecting sediments and sterilizing the sediments at 80° C. to 95° C. for 20 to 25 minutes; pre-freezing the sediments at −82° C. to −80° C. for 2 to 3 hours, and freeze-drying the sediments to obtain powers of the Bifidobacterium animalis subsp. lactis IOB-LO7 postbiotics, wherein a drying temperature is set to −80° C. to −60° C., and a drying time is 24 to 48 hours.

In a preferred embodiment, the pharmaceutical for protecting nerve cells includes the Bifidobacterium animalis subsp. lactis IOB-LO7, the Bifidobacterium animalis subsp. lactis IOB-LO7 powders, or the Bifidobacterium animalis subsp. lactis IOB-LO7 postbiotics.

In a preferred embodiment, a viable bacterial count of the Bifidobacterium animalis subsp. lactis IOB-LO7 powders is 1×10⁹ to 8×10¹¹ cfu/g, and a viable bacterial count of the pharmaceutical for protecting nerve cells is 1×10⁹ to 8×10¹¹ cfu/g.

To further elaborate on the technical methods and the effects employed in the present disclosure, the following describes the technical solution of the disclosure with reference to the preferred embodiments. However, it should be understood that the present disclosure is not limited to these embodiments.

Healthy and specific pathogen free (SPF) Wistar rats were randomly divided into five groups, with 10 rats in each group: a control group, a model group, a positive drug group (administered donepezil hydrochloride by gavage), an IOB-LO7 bacterial powders group, and an IOB-LO7 postbiotics group. The positive drug group, the IOB-LO7 bacterial powders group, and the IOB-LO7 postbiotics group are experimental groups. Except for the control group, the other four groups of rats received intraperitoneal injections of D-galactose (90 mg/kg/day) for 13 consecutive weeks, along with oral administration of AlCl3 (40 mg/kg/day) to establish an Alzheimer's disease model.

One hour after the intraperitoneal injection of D-galactose and oral gavage of AlCl3, the control and model groups were given sterile physiological saline (0.5 mL per rat) by gavage. The positive drug control group was administered donepezil hydrochloride (0.85 mg/kg/day, 0.5 mL per rat) by gavage. The IOB-LO7 bacterial powders group was given the corresponding dose of bacterial powder (1×10⁹ CFU/kg) by gavage, while IOB-LO7 postbiotics group was given the corresponding dose of postbiotics (1×10⁹ CFU/kg) by gavage. The gavage treatment continued for 3 months.

Testing embodiment 1: Bifidobacterium animalis subsp. lactis IOB-LO7 improves behavioral indicators in Alzheimer's disease rats.

### (1) Morris Water Maze Test

The Morris water maze test was conducted over five consecutive days, with training at four different starting entry points (in different quadrants) each day, as a navigation test. After the completion of the navigation test, removing the underwater platform 24 hours later. Placing the rats placed into the water at one of the starting entry point, and recording the number of times they crossed the platform within 60 seconds to assess their memory ability.

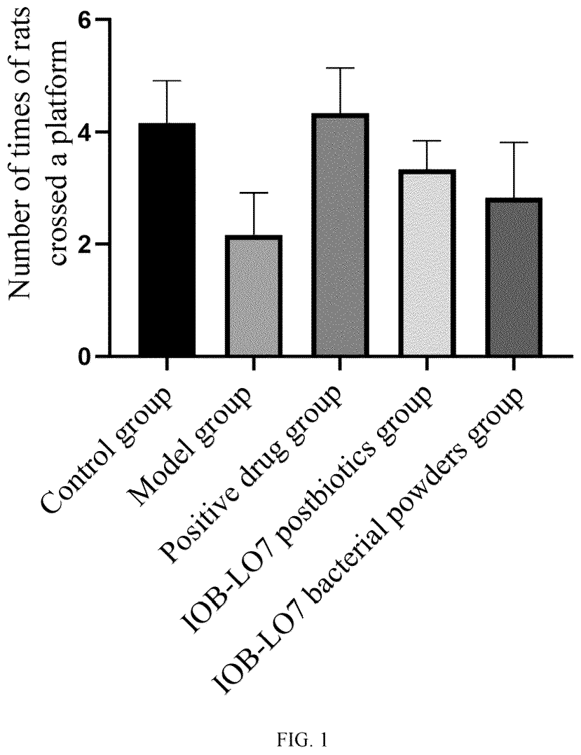

The results of the experiment are shown in FIG. 1. Compared to the control group, the model group rats showed a significant decrease in the number of platform crossings, indicating that the combination of aluminum chloride and D-galactose can impair memory. In comparison to the model group, the positive drug group, IOB-LO7 bacterial powders group, and IOB-LO7 postbiotics group showed an increase in the number of platform crossings.

### (2) Open Field Test (OFT)

The experimental apparatus consisted of four square boxes (0.8 m×0.8 m×0.5 m) made of plastic, with a trajectory recording device positioned above the boxes to track the rats' movements in real time. The total distance traveled, average speed, and number of activities in the boxes within 5 minutes were recorded.

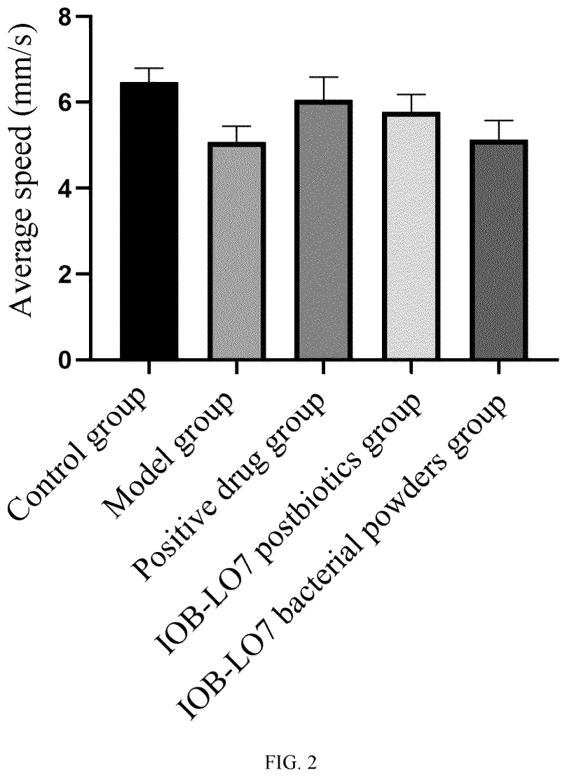

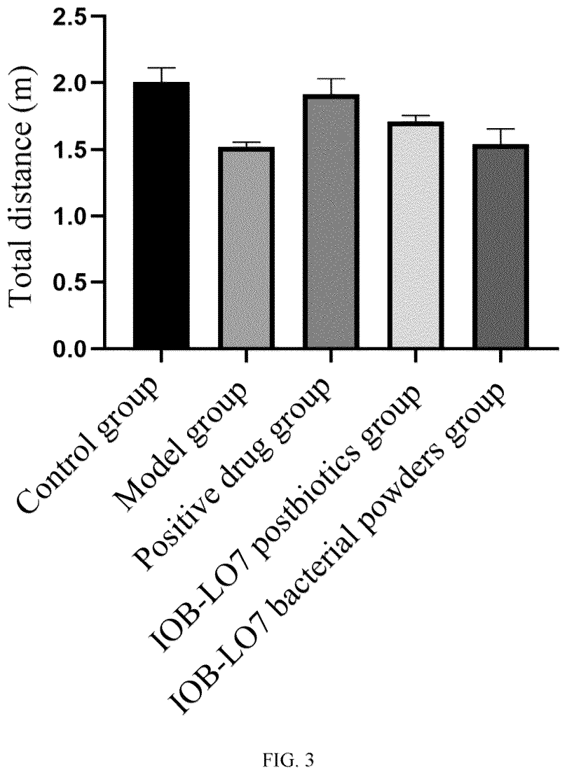

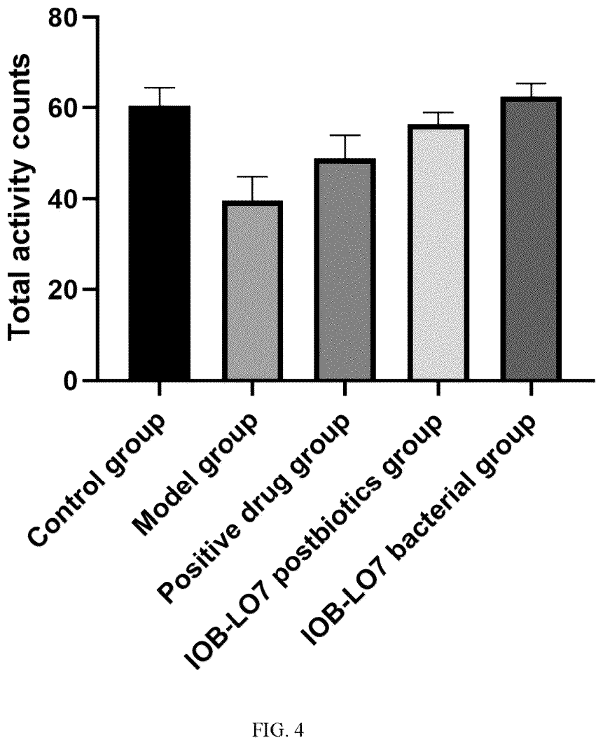

This experiment reflects the anxiety status of Alzheimer's disease animals based on their voluntary activity and exploratory behavior. The results are shown in FIGS. 2, 3, and 4. FIG. 2 presents the average speed of rats in the open field test. It shows that, compared to the control group, the model group rats had a significantly reduced average speed. In contrast, the positive drug group, IOB-LO7 bacterial powders group, and IOB-LO7 postbiotics group showed a significant increase in average speed compared to the model group. FIG. 3 shows the total distance traveled by rats in the open field test. Compared to the control group, the model group rats had a significantly reduced total distance. In comparison, the positive drug group, IOB-LO7 bacterial powders group, and IOB-LO7 postbiotics group had a significant increase in total distance traveled. FIG. 4 shows the number of activities performed by rats in the open field test. Compared to the control group, the model group rats had a significantly reduced number of activities. In contrast, the positive drug group, IOB-LO7 bacterial powders group, and IOB-LO7 postbiotics group have a significant increase in the number of activities compared to the model group.

In summary, the experimental results suggest that IOB-LO7 postbiotics can improve anxiety-like behaviors in D-galactose/AlCl3-induced Alzheimer's disease rats.

Testing embodiment 2: effect of Bifidobacterium animalis subsp. lactis IOB-LO7 on the Aβ42 concentration in the brains of Alzheimer's disease rats.

After 3 months of IOB-LO7 postbiotics and positive drug gavage treatment, the concentration of Aβ42 in rat brain samples was measured using the Aβ42 enzyme-linked immunosorbent assay (ELISA) kit.

Experimental materials include Aβ42 enzyme-linked immunosorbent assay (ELISA) kit, purchased from Nanjing Herbsource Biotechnology Co., Ltd.

Experimental procedure are as followed: coating the purified rat Aβ42 protein capture antibody on the microplate wells to form a solid-phase antibody. Adding the rat Aβ42 protein to the wells and allowed to bind with the HRP-labeled detection antibody. After thorough washing, adding TMB chromogenic reagent. Measuring optical density (OD value) at 450 nm using a microplate reader. Determining a concentration of Aβ42 in the rat brain samples a standard curve.

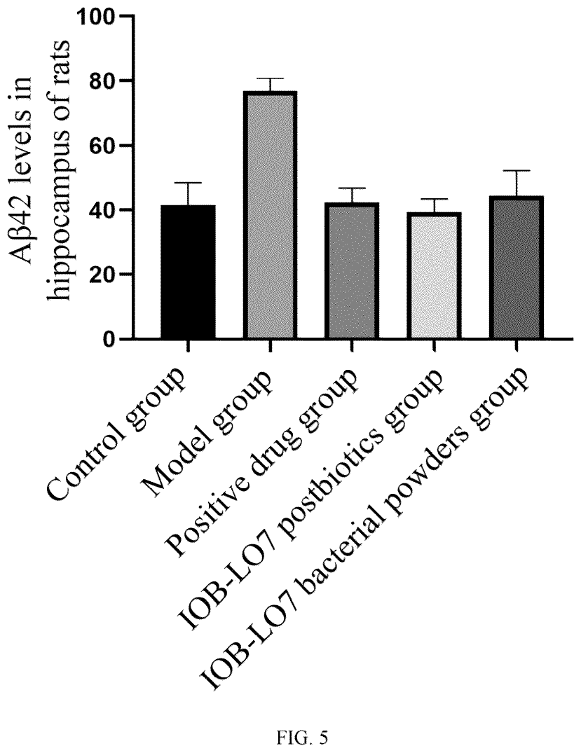

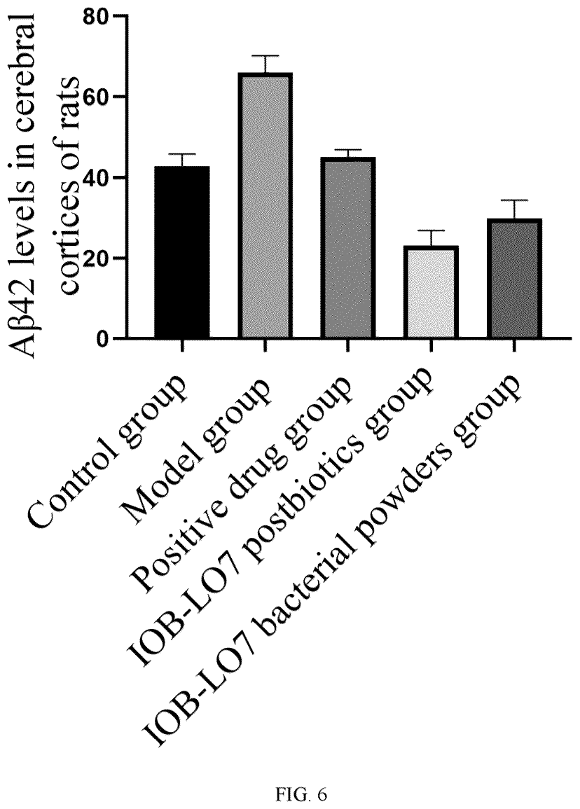

The experimental results are shown in Table 1, FIG. 5, and FIG. 6. The data from Table 1 represent the Aβ42 concentration in the brains of rats, with the average values calculated from multiple rat samples for each group. FIG. 5 shows the Aβ42 concentration in the hippocampus of rats. Compared to the control group, the model group exhibited a significant increase in Abeta42 deposition in the hippocampus (76.88 ng/L). In contrast, both the IOB-LO7 bacterial powders group and the postbiotics group showed a significant reduction in hippocampal Abeta42 deposition (44.57 ng/L and 39.50 ng/L, respectively). The percentage decrease in Aβ42 levels, calculated using the formula: decrease rate (%)=(model group value−experimental group value)/model group value×100%, and the decrease rate was 42.1% for the bacterial powders group, and the decrease rate was 48.6% for the postbiotics group.

FIG. 6 shows the Aβ42 concentration in the cerebral cortex. Compared to the control group, the model group exhibited a significant increase in Aβ42 concentration in the cerebral cortex (66.08 ng/L). In comparison to the model group, the IOB-LO7 bacterial powder group and the postbiotic group showed a significant reduction in Aβ42 levels in the cerebral cortex (29.96 ng/L and 23.23 ng/L, respectively). The decrease rate in Aβ42 levels was 54.6% for the bacterial powders group and 64.9% for the postbiotic group.

The above data suggest that Bifidobacterium animalis subsp. lactis IOB-LO7 can reduce the Aβ42 concentration in the brains of Alzheimer's disease rats.

TABLE 1
Aβ42 content in rat brain

| | groups | | | | |
|---|---|---|---|---|---|
| | | | positive | IOB-LO7 | IOB-LO7 |
| | control | model | drug | postbiotics | bacterial |
| | group | group | group | group | powders group |
| concentration | (ng/L) | (ng/L) | (ng/L) | (ng/L) | (ng/L) |
| Aβ42 | 41.53 | 76.88 | 42.38 | 44.57 | 39.50 |
| concentration | | | | | |
| in | | | | | |
| hippocampus | | | | | |
| Aβ42 | 42.93 | 66.08 | 45.17 | 29.96 | 23.23 |
| concentration | | | | | |
| in cerebral | | | | | |
| cortex | | | | | |

Testing embodiment 3: effect of the Bifidobacterium animalis subsp. lactis IOB-LO7 on the clearance of Aβ amyloid plaques in the brains of Alzheimer's disease rats.

The expression of Aβ42 in the hippocampus was observed using immunofluorescence labeling.

Experimental Materials include xylene, purchased from Shanghai Lingfeng Chemical Reagent Co., Ltd.; anhydrous ethanol, purchased from Tianjin Jiangtian Chemical Technology Co., Ltd.; EDTA (pH 8.0) antigen retrieval solution, PBS buffer solution, histology pens, BSA, primary antibody Iba˜1+A, DAPI, autofluorescence quenching reagent, anti-fluorescence quenching mounting medium, purchased from Wuhan Seville Biotechnology Co., Ltd.

Experimental process are as followed.
(1) Deparaffinization of paraffin sections: immersing the paraffin sections in xylene I solution for 15 minutes, and then immersing the paraffin sections in xylene II solution for 15 minutes. Then, placing the paraffin sections in anhydrous ethanol I for 5 minutes, and placing the paraffin sections in anhydrous ethanol II for 5 minutes. Afterward, immersing the paraffin sections in 85% and 75% ethanol for 5 minutes each, and finally washed with pure distilled water.
(2) Antigen retrieval: placing the paraffin sections in EDTA antigen retrieval solution (pH 8.0) and heating in a microwave. Once the solution was boiled, stopping the heating for 8 minutes, and then low heating for 7 minutes. Care was taken to prevent drying of the paraffin sections during heating. Naturally cooling the paraffin sections to room temperature, and performing a decolorizations for three times using PBS solution (pH 7.4).
(3) Serum blocking: when the sections were slightly dry, drawing a circle around the tissue using a histology pen. After drying the paraffin sections, adding BSA to the tissue for blocking for 30 minutes.
(4) Primary antibody incubation: after gently removing the blocking solution, adding the primary antibody (prepared with PBS) dropwise to the sections. Then incubated the paraffin sections overnight at 4° C. in a humidified box.
(5) DAPI staining for nuclear counterstaining: performing a decolorizations on the paraffin sections for three times with PBS (pH 7.4), then dried; adding DAPI solution dropwise to the paraffin sections, and incubating the paraffin sections at room temperature for 10 minutes in the dark.
(6) Quenching of autofluorescence: performing a decolorizations on the paraffin sections for three times with PBS (pH 7.4), then dried; adding autofluorescence quenching reagent to the tissue and incubating for 5 minute; and washing the paraffin sections with flowing water for 10 minutes.
(7) Mounting: after drying, an anti-fluorescence quenching mounting medium was added dropwise to mount the sections.
(8) Microscopic examination and photography: observing the prepared tissue sections under a fluorescence inverted microscope, to assess fluorescence intensity and capture fluorescence images.

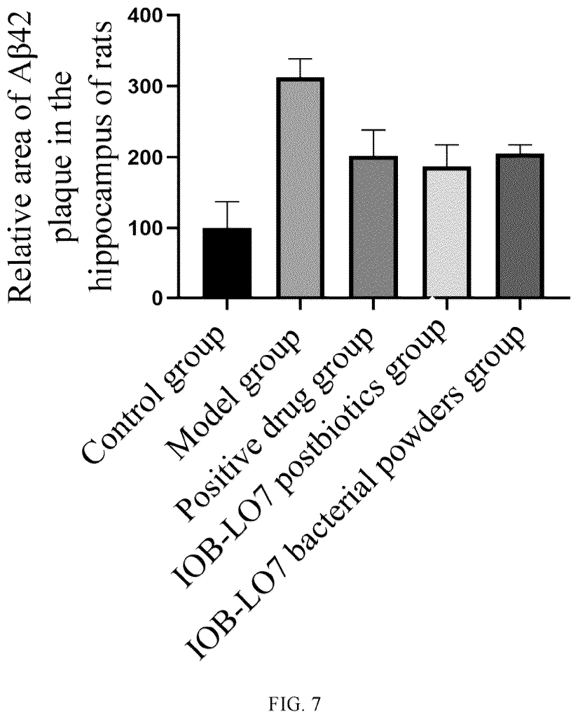

Results: as shown in FIG. 7, the control group exhibited minimal Aβ42 in the hippocampus. In contrast, the model group rats had a higher level of Aβ42 expression in the hippocampus, with numerous red plaque deposits in a fluorescence imaging of Aβ42 plaque deposition. Quantification by Image J analysis revealed a 313.1% increase in Aβ42. After 3 months of intervention, both the IOB-LO7 bacterial powders group and the postbiotics group showed a reduction in Aβ42 expression in the hippocampus. The area of Aβ42 plaques in the hippocampus significantly decreased, with quantification by Image J revealing reductions of 205.8% and 187.3%, respectively. According to the reduction rate formula, the reduction rate (%)=(model group value−experimental group value)/model group value×100%, the IOB-LO7 bacterial powders group showed a 34.3% reduction, and the IOB-LO7 postbiotics group showed a 40.2% reduction. The immunofluorescence results were consistent with the Aβ42 levels measured in the hippocampus using the ELISA kit.

Testing embodiment 4: inhibition of TLR4/NLRP3/MyD88 signaling pathway of the Bifidobacterium animalis subsp. lactis IOB-LO7 postbiotics and bacterial powders, to alleviate neuroinflammatory response.

Toll-like receptor 4 (TLR4) is a type I transmembrane receptor with the ability to sense both exogenous pathogens and endogenous inflammatory stimuli in the brain, including Aβ, LPS, and others. The TLR4/NLRP3/MyD88 signaling pathway can trigger an inflammatory response in the brain, promoting the transcriptional activity of nuclear factor kappa B and ultimately leading to the expression of pro-inflammatory cytokines such as IL˜1β and IL˜18. To observe the expression patterns of genes associated with the TLR4 signaling pathway and the activation levels of transcription factors, the relative transcription levels of NLRP3, MyD88, IL˜1β, and IL˜18 genes in rat brain tissue were measured.

Experimental materials include 2× universal SYBR green fast qPCR mix (5 mL), ABScript Neo RT master mix for qPCR with gDNA remover (100T), purchased from Wuhan Aibo Tech Biotechnology Co., Ltd.; and Trizol (total RNA extraction reagent, 100 mL), purchased from Tianjin Jiangtian Chemical Technology Co., Ltd.

Experimental Procedure are as followed.
(1) RNA Extraction and cDNA Synthesis: taking 50 to 100 mg of rat brain tissue, adding 1 mL of Trizol, and grinding rapidly to extract RNA; performing a reverse transcription to synthesize cDNA according to instructions of the ABScript Neo RT master mix for qPCR with gDNA remover kit.
(2) Real-time PCR reaction setup: preparing the reaction mixture using the 2× universal SYBR green fast qPCR mix kit; and caring an amplification by a real-time fluorescence quantitative PCR system. The primer information is provided in Table 2. The reaction conditions are as follows: 95° C. for 3 minutes (pre-denaturation), 95° C. for 15 seconds (denaturation), 60° C. for 30 seconds (annealing), and 40 cycles were performed. The relative mRNA transcription levels of the target genes were calculated using s 2^{-ΔΔCt} formula.

TABLE 2
Primer sequences for real-time fluorescence quantitative PCR

| gene | forward primer (5′ to 3′) | reverse primer (5′ to 3′) |
|---|---|---|
| TLR4 | GGCTTCTAACCTCAACGACCT | ATGATTCTTTGCCTGAGTTGC |
| | (SEQ ID NO. 2) | (SEQ ID NO. 3) |
| NLRP3 | CTGTGCTTAGAGACATGGGCA | AGGGACACTGGTTGCAGTAG |
| | (SEQ ID NO. 4) | (SEQ ID NO. 5) |
| MyD88 | TATACCAACCCTTGCACCAAGTC | CAGGCTCCAAGTCAGCTCATC |
| | (SEQ ID NO. 6) | (SEQ ID NO. 7) |
| IL~18 | CGGAGCATAAATGACCAAGTTCT | CCATTTTCATCCTTCCTCTTCAA |
| | (SEQ ID NO. 8) | (SEQ ID NO. 9) |
| IL~1β | TGCCTGGTCTTGTGACTTGGAG | TGTCCTGGGAGGAGGTAGAAACG |
| | (SEQ ID NO. 10) | (SEQ ID NO. 11) |
| β~actin | ACATCCGTAAAGACCTCTATGCC | TACTCCTGCTTGCTGATCCAC |
| | (SEQ ID NO. 12) | (SEQ ID NO. 13) |

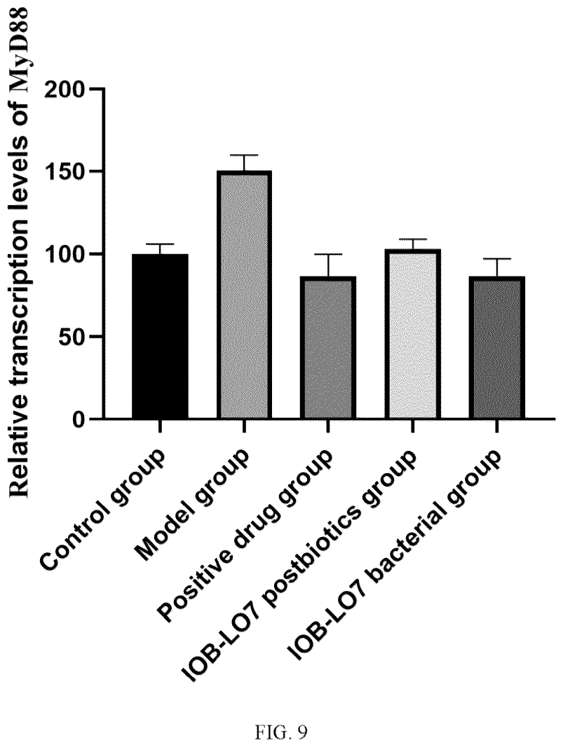

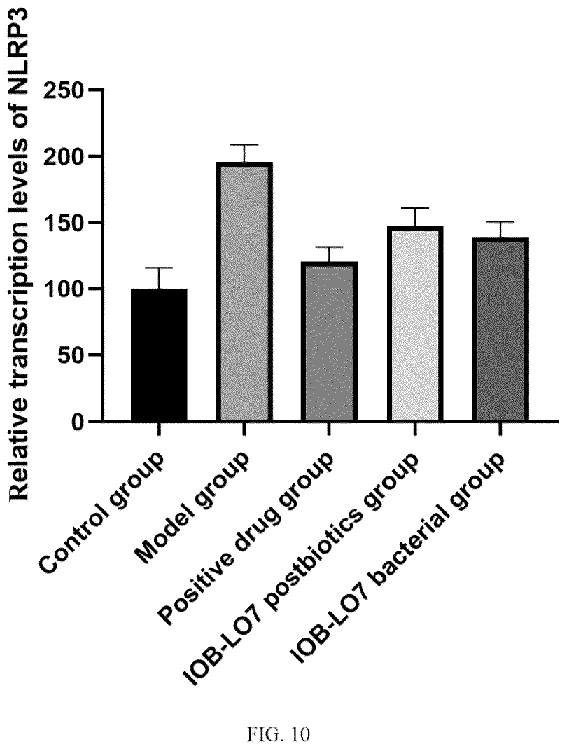

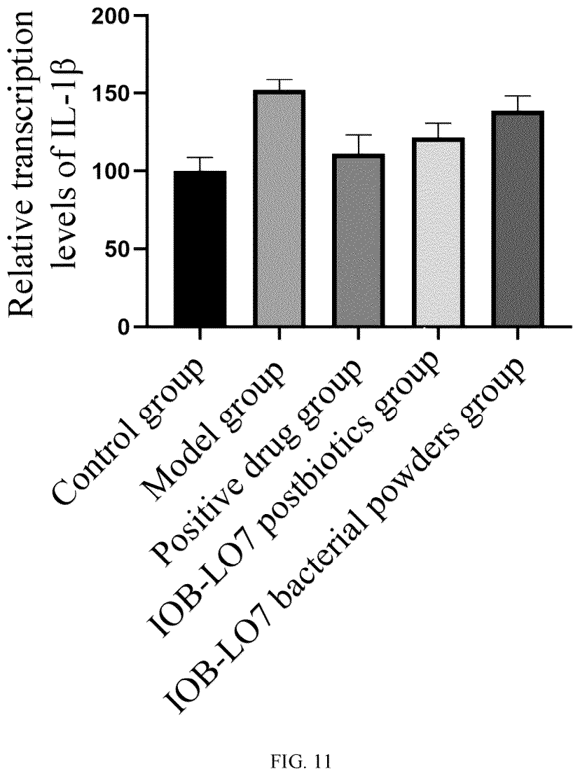

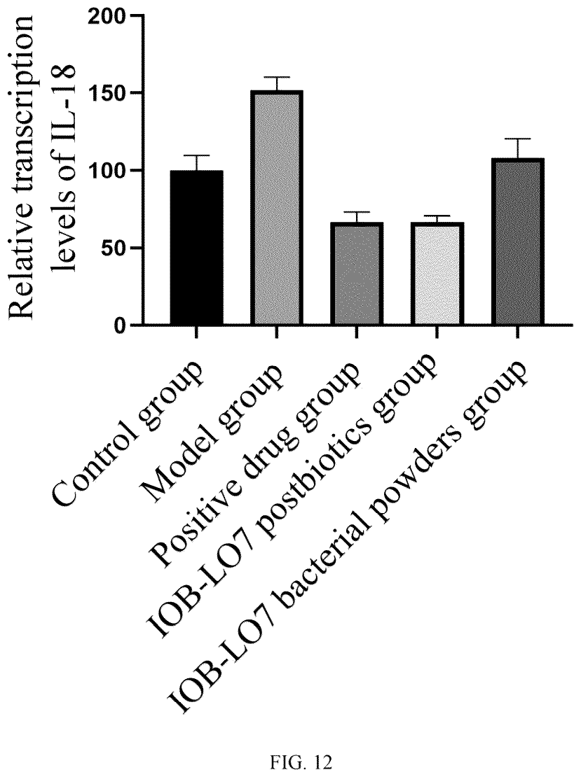

The results shown in FIGS. 9, 10, 11, 12, and 13 indicate that, compared with the control group, the relative expression levels of TLR4, NLRP3, MyD88, IL˜1β and IL˜18 were significantly increased in the model group. The positive drug group showed a significant difference from the model group, with a marked down regulation of the relative transcription levels of TLR4, NLRP3, MyD88, IL˜1β and IL˜18. Similarly, both the IOB-LO7 postbiotics group and the bacterial powders group exhibited significant down regulation of the expression of TLR4, NLRP3, MyD88, IL˜1β and IL˜18 related genes compared to the model group. Specifically, the expression levels of IL˜1β and IL˜18 genes in the IOB-LO7 postbiotics group decreased by 19.7% and 56.2%, respectively, while in the bacterial powders group, they decreased by 9.4% and 28.8%.

These results suggest that both the Bifidobacterium animalis subsp. lactis IOB-LO7 postbiotics and Bifidobacterium animalis subsp. lactis IOB-LO7 bacterial powders can inhibit the TLR4/MyD88/NLRP3 signaling pathway and alleviate neuroinflammation in Alzheimer's disease rats.

Testing embodiment 5: Bifidobacterium animalis subsp. lactis IOB-LO7 postbiotics reduce LPS levels in the gut contents, serum, and hippocampus of Alzheimer's disease rats.

Experimental material includes rat LPS ELISA kit, purchased from Nanjing Herbal Source Biotechnology Co., Ltd.

The rat LPS ELISA kit was configured to measure the levels of LPS in the gut contents, serum, and brain tissue of rats.

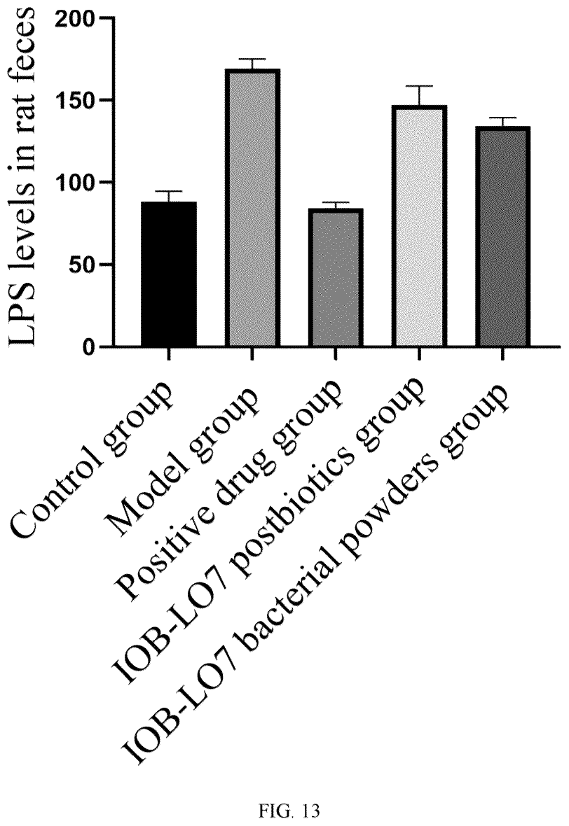

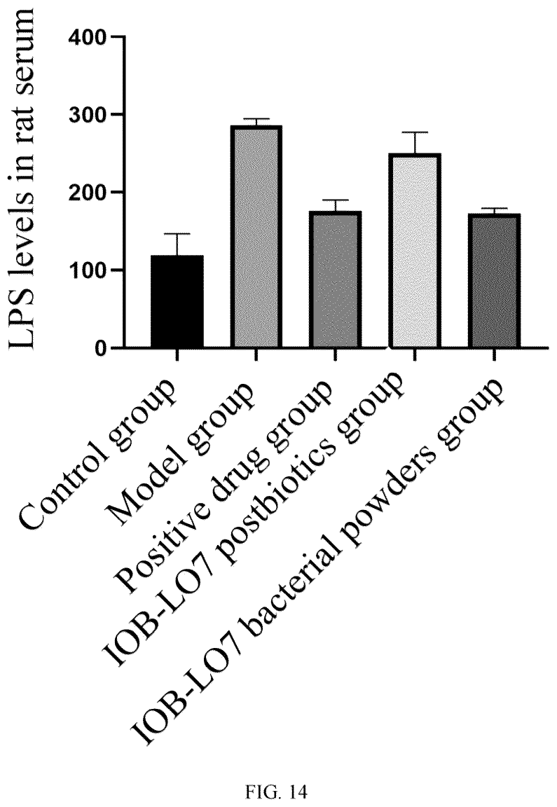

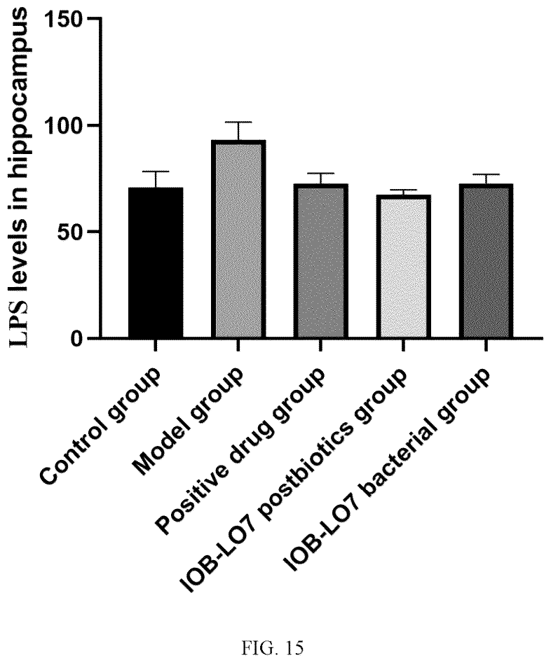

Results: FIG. 13 shows the LPS concentration in rat feces. Compared to the control group, the model group exhibited significantly higher LPS levels in the gut contents. In contrast, the positive drug group, IOB-LO7 bacterial powders group, and IOB-LO7 postbiotics group all showed a decreasing trend in LPS concentration when compared to the model group. FIG. 14 illustrates the LPS levels in the serum of rats from each group. It can be seen that, compared to the control group, the model group had significantly higher LPS levels in the serum. Furthermore, the positive drug group, IOB-LO7 bacterial powders group, and IOB-LO7 postbiotic group all showed significant reductions in serum LPS levels compared to the model group. FIG. 15 shows the LPS concentration in the hippocampus of rats in each group. Compared to the control group, the model group had significantly higher LPS levels in the brain tissue. In contrast, the positive drug group, IOB-LO7 bacterial powders group, and IOB-LO7 postbiotics group showed a clear reduction in LPS levels in the brain tissue.

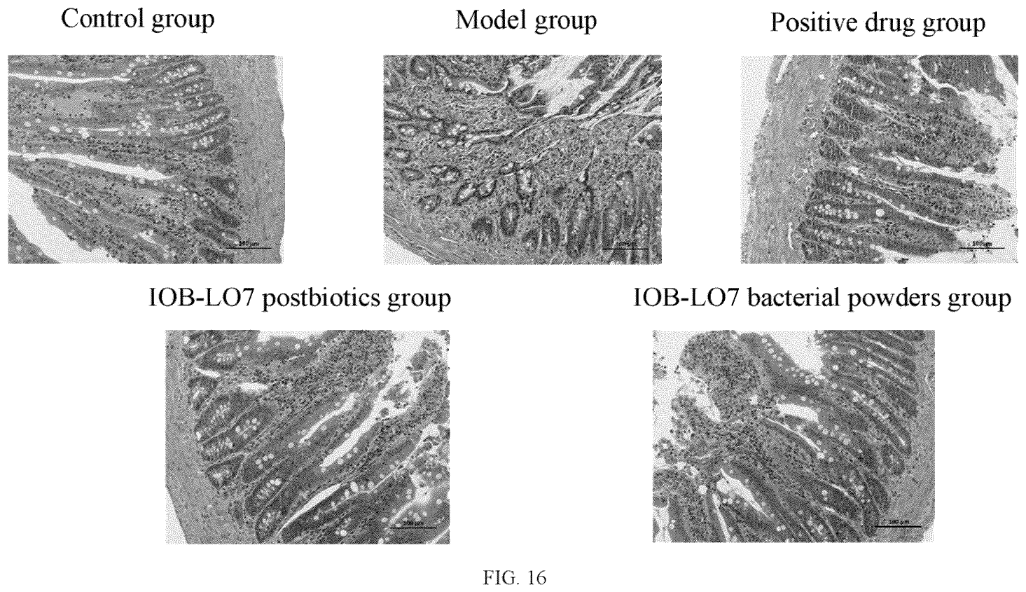

Further analysis: the pathological sections of the rat small intestine were analyzed to observe changes in the morphology of intestinal tissue. As shown in FIG. 16, compared to the control group, the small intestinal villi of rats in the model group were significantly shortened or even atrophied, sparsely arranged, and with disorganized tissue structure. After intervention with IOB-LO7 bacterial powders and IOB-LO7 postbiotics, although there was some villus shedding at the top of the colon in all groups, the length of the villi at the bottom of the colon increased, and the tissue structure appeared more organized. These results suggest that postbiotics can partially improve the morphology of the small intestine in Alzheimer's disease rats, effectively maintain the intestinal mucosal structure, and offer some protective effects against intestinal tissue damage.

In conclusion, supplementation with IOB-LO7 bacterial powders or postbiotics can alleviate gut mucosal barrier damage in Alzheimer's disease rats, preventing the release and spread of harmful metabolites such as LPS.

The above are only some embodiments of the present disclosure, and neither the words nor the drawings can limit the protection scope of the present disclosure. Any equivalent structural transformation made by using the contents of the specification and the drawings of the present disclosure under the overall concept of the present disclosure, or directly/indirectly applied in other related technical fields are included in the protection scope of the present disclosure.

What is claimed is:

1. A Bifidobacterium animalis subsp. lactis for reducing Abeta42 deposition, wherein, a name of the Bifidobacterium animalis subsp. lactis is Bifidobacterium animalis subsp. lactis IOB-LO7, and classified as Bifidobacterium animalis subsp. lactis, the Bifidobacterium animalis subsp. lactis IOB-LO7 is preserved in the General Microbiology Center of the China General Microbiological Culture Collection Center (CGMCC) on Dec. 23, 2021, with the collection number of CGMCC No. 24185.

2. An application of a Bifidobacterium animalis subsp. lactis IOB-LO7 for reducing Abeta42 deposition, wherein, the Bifidobacterium animalis subsp. lactis being applied for preparing Bifidobacterium animalis subsp. lactis IOB-LO7 bacterial powders, Bifidobacterium animalis subsp. lactis IOB-LO7 postbiotics, or pharmaceutical for protecting nerve cells.

3. The application of the Bifidobacterium animalis subsp. lactis IOB-LO7 for reducing Abeta42 deposition according to claim 2, wherein, the Bifidobacterium animalis subsp. lactis bacterial powders are prepared by the following steps:
preparing fermentation broth; and
centrifuging the fermentation broth at a speed of 4000 to 6000 rpm for 20 to 30 minutes, wherein a temperature of the centrifuge is reduced to 4° C. to 6° C. in advance; and
collecting sediments and pre-freezing the sediments at −82° C. to −80° C. for 2 to 3 hours; and
performing a freeze-drying at a temperature of −80° C. to −60° C. for 24 to 48 hours to obtain the Bifidobacterium animalis subsp. lactis IOB-LO7 postbiotics powders.

4. The application of the Bifidobacterium animalis subsp. lactis IOB-LO7 for reducing Abeta42 deposition according to claim 2, wherein the Bifidobacterium animalis subsp. lactis IOB-LO7 postbiotics are prepared by the following steps:
preparing the fermentation broth; and
centrifuging the fermentation broth at a speed of 4000 to 6000 rpm for 20 to 30 minutes, wherein a temperature of the centrifuge is reduced to 4° C. to 6° C. in advance; and
collecting sediments and sterilizing the sediments at 80° C. to 95° C. for 20 to 25 minutes;
pre-freezing the sediments at −82° C. to −80° C. for 2 to 3 hours, and freeze-drying the sediments to obtain powers of the Bifidobacterium animalis subsp. lactis IOB-LO7 postbiotics, wherein a drying temperature is set to −80° C. to −60° C., and a drying time is 24 to 48 hours.

5. The application of the Bifidobacterium animalis subsp. lactis IOB-LO7 for reducing Abeta42 deposition according to claim 3, wherein the fermentation broth is prepared by the following steps:
inoculating the Bifidobacterium animalis subsp. lactis IOB-LO7, stored in cryovials, into 15 to 20 mL of TPY fluid medium; and culturing for 18 to 24 hours in a strictly anaerobic environment at a culturing temperature of 35° C. to 38° C., to obtain first generation activated bacterial solution; and
inoculating the first generation activated bacterial solution into 50 to 60 mL of the TPY fluid medium at an inoculation rate of 3% to 5%; and culturing for 18 to 24 hours in the strictly anaerobic environment at the culturing temperature of 35° C. to 38° C., to obtain second generation activated bacterial solution; and
inoculating the second generation activated bacterial solution into 90 to 100 mL of the TPY fluid medium at an inoculation rate of 1% to 3%; and culturing for 18 to 20 hours in the strictly anaerobic environment at the culturing temperature of 35° C. to 38° C., to obtain fermented seed liquid;
inoculating the fermented seed liquid into 800 to 1000 ml of the TPY fluid medium at the inoculation rate of 3% to 5%; and culturing for 18 to 20 hours in the strictly anaerobic environment at the culturing temperature of 35° C. to 38° C., to obtain the fermentation broth.

6. The application of the Bifidobacterium animalis subsp. lactis IOB-LO7 for reducing Abeta42 deposition according to claim 2, wherein the pharmaceutical for protecting nerve cells comprises the Bifidobacterium animalis subsp. lactis IOB-LO7, the Bifidobacterium animalis subsp. lactis IOB-LO7 powders, or the Bifidobacterium animalis subsp. lactis IOB-LO7 postbiotics.

7. The application of the Bifidobacterium animalis subsp. lactis IOB-LO7 for reducing Abeta42 deposition according to claim 2, wherein a viable bacterial count of the Bifidobacterium animalis subsp. lactis IOB-LO7 powders is 1×10⁹ to 8×10¹¹ cfu/g, and a viable bacterial count of the pharmaceutical for protecting nerve cells is 1×10⁹ to 8×10¹¹ cfu/g.
